SEQUENCE LISTING (5 sequences)

SEQ ID NO: 1 - protein, 151 aa, Homo sapiens
  misc_feature: Human Neuroglobin
MERPEPELIRQSWRAVSRSPLEHGTVLFARLFALEPDLLPLFQYNCRQFSSPEDCLSSPEFLDHIRKVMLVIDAAVTNVEDLSSLEEYLASLGRKHRAVGVKLSSFSTVGESLLYMLEKCLGPAFTPATRAAWSQLYGAVVQAMSRGWDGE

SEQ ID NO: 2 - protein, 155 aa, Mus musculus
  misc_feature: Mouse Neuroglobin
MERPESELIRQSWRVVSRSPLEHGTVLFARLFALEPSLLPLFQYNGRQFSSPEDCLSSPEFLDHIRKVMLVIDAAVTNVEDLSSLEEYLTSLGRKHRAVGVRLSSFSVGSGTVGESLLYMLEKCLGPDFTPATRTAWSRLYGAVVQAMSRGWDGE

SEQ ID NO: 3 - DNA, 19 bp, Artificial Sequence
  Forward primer - Example 4
ccctatctatgtgtgtctg

SEQ ID NO: 4 - DNA, 18 bp, Artificial Sequence
  Reverse primer - Example 4
tgaggaccaaggtataga

SEQ ID NO: 5 - DNA, 25 bp, Artificial Sequence
  Probe - Example 4
atctgcctgttgtagtcttagcctc

(19) United States
(12) Patent Application Publication
(10) Pub. No.: US 2024/0307339 A1
(43) Pub. Date: Sep. 19, 2024

(54) NOVEL COMPOUNDS FOR THE TREATMENT AND PREVENTION OF NEUROLOGICAL COMPLICATIONS OF VIRAL INFECTIONS

(71) Applicant: Sunregen Healthcare AG, Reinach (CH)

(72) Inventor: Yuhong Dong, Reinach (CH)

(21) Appl. No.: 18/571,123

(22) PCT Filed: Jun. 17, 2022
(86) PCT No.: PCT/EP2022/066548

§ 371 Date: Dec. 15, 2023

(30) Foreign Application Priority Data
Jun. 18, 2021 (EP) 21180462.0

(51) Int. Cl. A61K 31/23; A61K 36/12; A61P 25/28

(52) U.S. Cl. CPC A61K 31/23; A61K 36/12; A61P 25/28

(57) ABSTRACT

The present invention relates to a compound of formula (I):

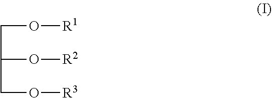

as well as a metabolite of the compound of formula (I), pharmaceutical composition comprising the compound of formula (I) or its metabolite, and/or ophioglossum for use in the treatment, prevention and/or alleviation of symptoms of neurological complication(s) of a viral infection disease. The instant compounds, metabolites, pharmaceutical compositions and ophioglossus are particularly useful in the treatment, prevention and/or alleviation of symptoms of neurological complication(s) of a viral infection disease caused by SARS-CoV-2.

### FIELD OF THE INVENTION

The present invention relates to a compound of formula (I):

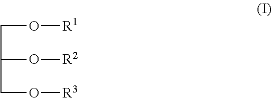

as well as a metabolite of the compound of formula (I), pharmaceutical composition comprising the compound of formula (I) or its metabolite, and/or ophioglossum for use in the treatment, prevention and/or alleviation of symptoms of neurological complication(s) of a viral infection disease. The instant compounds, metabolites, pharmaceutical compositions and ophioglossum are particularly useful in the treatment, prevention and/or alleviation of symptoms of neurological complication(s) of a viral infection disease caused by SARS-CoV-2.

### BACKGROUND OF THE INVENTION

The onset of neurological disorders has been connected to viral infections, which may cause neurological symptoms or lead to immune responses that trigger these pathological signs. Currently, this relationship is mostly based on epidemiological data on infections and seroprevalence of patients who present with neurological disorders. Neurologic manifestations including seizures, status epilepticus, encephalitis, critical illness neuromyopathy, acute disseminated encephalomyelitis, acute necrotizing encephalitis, Guillan-Barré syndrome, transverse myelitis, and acute flaccid myelitis have all been associated with severe viral respiratory infections.

Of particular attention is viral infection disease caused by SARS-CoV-2 virus, which is causing a global medical emergency that is taking a substantial number of lives every day. While the principal complication of coronavirus disease 2019 (COVID-19) caused by SARS-CoV-2 virus is respiratory failure, a considerable number of patients have been reported with neurological symptoms affecting both the peripheral and central nervous systems Neurological symptoms including headache, anosmia, ageusia, confusion, seizure, and encephalopathy have been frequently reported in COVID-19 patients, and SARS-CoV-2 RNA has been detected in brain biopsies of over 30% of fatal COVID-19 cases. SARS-CoV-2 infection has been postulated to lead, in the long term, to accelerated aging phenotypes in survivors in a range of tissues, including brain. It has been postulated that the chronic and long-term consequences of SARS-CoV-2 infection in the CNS should be closely monitored, also when the pandemic is over. Of note, several other viruses beyond SARS-CoV-2 are associated with major brain disorders like Alzheimer's, Parkinson's and multiple sclerosis' disease.

To date, no therapies for treatment, prevention and/or alleviation of symptoms of neurological complication(s) of a viral infection disease, in particular wherein the viral infection disease is caused by SARS-CoV-2, is known.

### SUMMARY OF THE INVENTION

It was an objective technical problem of the present invention to provide novel compounds for use in the treatment of neurological complication(s) of a viral infection disease, in particular wherein the viral infection disease is caused by SARS-CoV-2.

The objective technical problem is solved by the embodiments presented herein and as characterized by the claims.

The present inventors have surprisingly found that in the mouse model of coronavirus infection the treatment with the compound of the formula (I) leads to an improved clinical score in comparison with the treatment with the vehicle, as well as reduced weight loss of animals (see Example 1 and FIG. 1 for details). The compound of formula (I) has further shown the surprising ability to rescue the N-nitroso-N-methyl-urea induced loss of neuronal cells in the mouse models (Example 2, FIG. 2). The present inventors have further linked treatment with the compound of formula (I) with the induced increased expression of neuroglobin in neuronal cells, which is considered to be an unexpected result (Example 4, FIG. 4). Of note, this unexpected observation allowed the use of the compound of formula (I) for modulating expression of neuroglobin in neuronal cells of a subject in order to treat, prevent and/or alleviate the symptoms of neurological complication(s) of a viral infection disease, as encompassed by the present invention.

The present invention is summarized in the following embodiments.

In a first embodiment, the present invention relates to a compound of the formula (I)

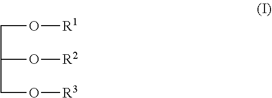

wherein R¹, R² and R³ are independently selected from H or —C(O)—C₁₄-alkyl, provided that at least one of R¹, R² and R³ is —C(O)—C₁₄-alkyl, for use in the treatment, prevention and/or alleviation of symptoms of neurological complication(s) of a viral infection disease.

In a particular embodiment, the present invention relates to the compound for use of the formula (I), wherein R¹, R² or R³ is —C(O)—C₁₄-alkyl.

In a further particular embodiment, the present invention relates to the compound for use of the formula (I), wherein any two of R¹, R² and R³ are —C(O)—C₁₄-alkyl.

In again further particular embodiment, the present invention relates to the compound for use of the formula (I), wherein R¹, R² and R³ are —C(O)—C₁₄-alkyl.

In again further particular embodiment, the present invention relates to the compound for use of the formula (I), wherein in the compound is tripentadecanoin.

In a further embodiment, the present invention relates to a metabolite of the compound of formula (I), wherein the metabolite is HO—C(O)—C₁₄-alkyl or a pharmaceutically acceptable salt thereof, for use in the treatment, prevention and/or alleviation of symptoms of neurological complication(s) of a viral infection disease.

In again a further embodiment, the present invention relates to a pharmaceutical composition comprising the compound of formula (I) or the metabolite of the compound of formula (I), and a pharmaceutically acceptable carrier, for use in the treatment, prevention and/or alleviation of symptoms of neurological complication(s) of a viral infection disease.

In again a further embodiment, the present invention relates to ophioglossum for use in the treatment, prevention and/or alleviation of symptoms of neurological complication(s) of a viral infection disease.

In a particular embodiment, the present invention relates to the compound for use of the present invention, the metabolite for use of the present invention, the pharmaceutical composition for use of the present invention, or the ophioglossum for use of the present invention, wherein the viral infection disease is caused by SARS-CoV-2, SARS-CoV-1, MERS, influenza virus, human immunodeficiency virus (HIV), varicella-zoster virus (VZV), herpes simplex virus (HSV), poliovirus, Epstein-Barr virus (EBV), cytomegalovirus (CMV), Japanese Encephalitis virus, Venezuelan Equine Encephalitis virus, California encephalitis virus or zika virus.

In a further particular embodiment, the present invention relates to the compound for use of the present invention, the metabolite for use of the present invention, the pharmaceutical composition for use of the present invention, or the ophioglossum for use of the present invention, wherein the viral infection disease is caused by SARS-CoV-2, SARS-CoV-1, or MERS.

In again a further particular embodiment, the present invention relates to the compound for use of the present invention, the metabolite for use of the present invention, the pharmaceutical composition for use of the present invention, or the ophioglossum for use of the present invention, wherein the viral infection disease is caused by SARS-CoV-2.

In again a further particular embodiment, the present invention relates to the compound for use of the present invention, the metabolite for use of the present invention, the pharmaceutical composition for use of the present invention, or the ophioglossum for use of the present invention, wherein the neurological complication of a viral infection disease is damage of the central nervous system, in particular of brainstem.

In again a further particular embodiment, the present invention relates to the compound for use of the present invention, the metabolite for use of the present invention, the pharmaceutical composition for use of the present invention, or the ophioglossum for use of the present invention, wherein symptoms of the damage of the central nervous system, in particular of brainstem, are difficulties in breathing and/or in heartbeat.

In again a further particular embodiment, the present invention relates to the compound for use of the present invention, the metabolite for use of the present invention, the pharmaceutical composition for use of the present invention, or the ophioglossum for use of the present invention, wherein the neurological complication(s) of a viral infection disease comprise damage of the peripheral nervous system.

In again a further particular embodiment, the present invention relates to the compound for use of the present invention, the metabolite for use of the present invention, the pharmaceutical composition for use of the present invention, or the ophioglossum for use of the present invention, wherein symptoms of the damage of the peripheral nervous system are muscle weakness, loss of taste and/or smell, limb pain, and/or malaise.

In again a further particular embodiment, the present invention relates to the compound for use of the present invention, the metabolite for use of the present invention, the pharmaceutical composition for use of the present invention, or the ophioglossum for use of the present invention, wherein symptoms of the neurological complication(s) of a viral infection disease are headache and/or seizures.

In again a further particular embodiment, the present invention relates to the compound for use of the present invention, the metabolite for use of the present invention, the pharmaceutical composition for use of the present invention, or the ophioglossum for use of the present invention, wherein preventing neurological complication(s) of a viral infection disease is preventing an onset of neurodegenerative diseases such as Alzheimer's disease or Parkinson's disease.

In again a further particular embodiment, the present invention relates to the compound for use of the present invention, the metabolite for use of the present invention, the pharmaceutical composition for use of the present invention, or the ophioglossum for use of the present invention, wherein preventing the neurological complication(s) of a viral infection disease constitutes preventing cardiorespiratory failure.

In again a further particular embodiment, the present invention relates to the compound for use of the present invention, the metabolite for use of the present invention, the pharmaceutical composition for use of the present invention, or the ophioglossum for use of the present invention, wherein the neurological complication(s) of a viral infection disease comprises perivascular encephalitis, interstitial encephalitis, neuronal cell loss, and/or axon degeneration.

In a further particular embodiment, the present invention relates to the compound for use of the present invention, the metabolite for use of the present invention, the pharmaceutical composition for use of the present invention, or the ophioglossum for use of the present invention, wherein the compound of formula (I) upon administration to the subject upregulates expression of neuroglobin, preferably in neuron cells.

In again a further particular embodiment, the present invention relates to the compound for use of the present invention, the metabolite for use of the present invention, the pharmaceutical composition for use of the present invention, or the ophioglossum for use of the present invention, wherein upregulated expression of neuroglobin inhibits apoptosis of neuron cells.

In again a further particular embodiment, the present invention relates to the compound for use of the present invention, the metabolite for use of the present invention, the pharmaceutical composition for use of the present invention, or the ophioglossum for use of the present invention, wherein the compound of formula (I) is to be administered in a dosage from 1 mg/day to 1000 mg/day.

### BRIEF DESCRIPTION OF FIGURES

FIG. 1: A,B: Clinical score comparison for treatment with vehicle and with the compound of formula (I) (herein, tripentadecanoin) of the mouse coronavirus infection model; C,D. weight loss comparison for treatment with vehicle and with the compound of formula (I) (herein, tripentadecanoin) of the mouse coronavirus infection model.

FIG. 2: Tripentadecanoin, a representative compound of the compound of formula (I) shows neuroprotective effects in the murine model of N-nitroso-N-methylurea photoreceptor degeneration.

FIG. 3: A. Representative images of a young cell, or old cells untreated or treated with Herb B—Ophioglossum thermale (10 μg/ml) or SBC003-tripentadecanoin (30 μM). Upper panels show cells stained with fluorescent brightener 28 to reveal bud scars. Lower panels display the Hsp104-GFP signal. The arrow points at an age-induced protein deposit. B. Percentage of cells with an Hsp104-GFP focus. Mean±SD. P values are adjusted p values from an ANOVA comparing to vehicle only. C. Average fluorescence intensity of cytoplasmic Hsp104-GFP. Mean±SD. P values are adjusted p values obtained from ANOVA comparing to vehicle only. D. Hsp104-GFP foci fluorescent intensities are comparable in all conditions. Mean±SD.

FIG. 4: A: Yeast genes whose expression was upregulated upon treatment with the compound of formula (I) (herein, tripentadecanoin) in comparison to vehicle; B: Images of old cells untreated or treated with tripentadecanoin. Bud scars stained with FB28 (upper panels). Hsp104-GFP fluorescence signal (lower panels). Arrow point at age-induced protein deposits. B. Percentage of cells with an Hsp104-GFP focus. Mean±SD. P values are adjusted p values from an ANOVA comparing to wild type control (black stars) or to wild type treated with tripentadecanoin (light grey stars). **<0.01; ****<0.0001; ns=not significantly different.

FIG. 5: An effect of treatment with the compound of formula (I) (herein, tripentadecanoin) on expression of neuroglobin in mouse primary cortex neurons.

### DETAILED DESCRIPTION OF THE INVENTION

The compounds for use of the present invention will be described in the following. It is to be understood that all possible combinations of the following definitions are also envisaged.

In one embodiment, the present invention relates to a compound of the formula (I)

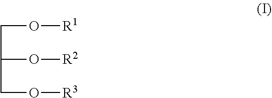

for use in the treatment, prevention and/or alleviation of symptoms of neurological complication(s) of a viral infection disease. R¹, R² and R³ are independently selected from H or —C(O)—C₁₄-alkyl, provided that at least one of R¹, R² and R³ is —C(O)—C₁₄-alkyl.

In one embodiment, R¹, R² or R³ is —C(O)—C₁₄-alkyl. In other words, one of R¹, R² and R³ is —C(O)—C₁₄-alkyl and two remaining of R¹, R² and R³ are H.

In a further embodiment, any two of R¹, R² and R³ are —C(O)—C₁₄-alkyl. That further means that one of R¹, R² and R³ is H.

Preferably, R¹, R² and R³ are —C(O)—C₁₄-alkyl. Further preferably, the compound of formula (I) is tripentadecanoin. Tripentadecanoin, as understood herein, is the compound of formula (I) wherein R¹, R² and R³ are —C(O)-tetradec-1-yl.

The compounds of formula (I) wherein at least one of R¹, R² and R³ is H may also be understood herein as metabolites of the compound of formula (I), wherein R¹, R² and R³ are —C(O)—C₁₄-alkyl. As it is understood by the skilled person, upon administration to a subject, preferably to a human subject, the ester bonds within the glyceride moiety of the compound of the formula (I) may hydrolyze, for example in a reaction catalyzed by an enzyme, yielding another glyceride encompassed by formula (I), or glycerol, and a carboxylic acid according to formula HO—C(O)—C₁₄-alkyl (or a salt thereof, in particular a pharmaceutically acceptable salt thereof). The said carboxylic acid may also be referred to as a metabolite of the compound of formula (I).

Hence, in a further embodiment, the present invention relates to a metabolite of the compound of formula (I), for use in the treatment, prevention and/or alleviation of symptoms of neurological complication(s) of a viral infection disease. Preferably, the metabolite of the compound of formula (I) is HO—C(O)—C₁₄-alkyl or a pharmaceutically acceptable salt thereof. In a particular embodiment, wherein the compound of formula (I) is tripentadecanoin, the said metabolite HO—C(O)—C₁₄-alkyl or a pharmaceutically acceptable salt thereof is n-pentadecanoic acid or a pharmaceutically acceptable salt thereof.

The compounds of the present invention and/or the metabolites of the compounds of the present invention can be administered to a patient in the form of a pharmaceutical composition which can optionally comprise one or more pharmaceutically acceptable excipient(s) and/or carrier(s). Thus, the present invention relates to a pharmaceutical composition comprising the compound of the present invention or the metabolite of the compound of the present invention, and a pharmaceutically acceptable carrier, for use in the treatment, prevention and/or alleviation of symptoms of neurological complication(s) of a viral infection disease

Depending on the route of administration the compound of the invention or the metabolite of the invention can be provided in various pharmaceutical formulations. Some of the said pharmaceutical formulations may require that protective coatings are applied to the formulation to prevent degradation of the compound of the invention or of the metabolite of the invention in, for example, the digestive tract. The compound of the invention (or the metabolite of the invention) can be formulated as a syrup, an infusion solution, injection solution, a spray, a tablet, a capsule, a capslet, a lozenge, a liposome, a suppository, a plaster, a band-aid, a retard capsule, a powder, or a slow release formulation.

The compound of the present invention or the metabolite of the compound of the present invention is preferably administered orally. Therefore, particular preferred pharmaceutical forms for the administration of the compound or the metabolite of the compound of the present invention are forms suitable for oral administration. Formulations for oral administration are usually supplied in dosage units and may contain conventional excipients, such as binders, fillers, diluents, tableting agents, lubricants, detergents, disintegrants, colorants, flavors and wetting agents. Tablets may be coated in accordance to methods well known in the art. Suitable fillers include or are preferably cellulose, mannitol, lactose and similar agents. Suitable disintegrants include or are preferably starch, polyvinyl pyrrolidone and starch derivatives such as sodium starch glycolate. Suitable lubricants include or are preferably, for example, magnesium stearate. Suitable wetting agents include or are preferably sodium lauryl sulfate. These solid oral compositions can be prepared with conventional mixing, filling or tableting methods. The mixing operations can be repeated to disperse the active agent in compositions containing large quantities of fillers. These operations are known to the skilled person.

According to the present disclosure, the pharmaceutical compositions comprising the compound of the invention or the metabolite of the compound of the present invention as liquid compositions for oral administration can be provided in the form of, for example, aqueous solutions, emulsions, syrups or elixirs or in the form of a dry product to be reconstituted with water or with a suitable liquid carrier at the time of use. The liquid compositions can contain conventional additives, such as suspending agents, for example sorbitol, syrup, methylcellulose, gelatin, hydroxyethyl cellulose, carboxymethyl cellulose, aluminum stearate gel or hydrogenated edible fats; emulsifying agents, for example lecithin, sorbitan monooleate, or acacia; non aqueous carriers (which can include edible oil), for example almond oil, fractionated coconut oil, oily esters, such as glycerin esters, propylene glycol or ethyl alcohol; preservatives, for example methyl or propyl p-hydroxybenzoate or sorbic acid; penetration enhancer, for example dimethylsulfoxide (DMSO); pH buffer systems, for example phosphate buffer, carbonate buffer, citrate buffer, citrate-phosphate buffer and other pharmaceutically acceptable buffer systems; solubilizers, for example beta-cyclodextrin, and if desired, conventional flavors or colorants.

Oral formulations may optionally further include taste-masking components to optimize the taste) perception of the oral formulation. Examples of such taste-masking components may be citrus-, licorice-, mint-, grape-, black currant-or eucalyptus-based flavorants known to those well-skilled in the art.

Further preferred forms of administration of the compounds of the present invention or of the metabolites of the compounds of the present invention include sterile aqueous solutions or dispersions and sterile powders for the extemporaneous preparation of sterile injectable solutions or dispersion. In all cases the final solution or dispersion form must be sterile and fluid. Sterilization of infusion or injection solutions can be accomplished by any number of art recognized techniques including but not limited to addition of preservatives like anti-bacterial or anti-fungal agents, e.g., parabene, chlorobutanol, phenol, sorbic acid or thimersal. Further, isotonic agents, such as sugars or salts, in particular sodium chloride may be incorporated in infusion or injection solutions.

Typically, such a solution or dispersion will include a solvent or dispersion medium, containing, for example, water-buffered aqueous solutions, e.g., biocompatible buffers (e.g., citrate buffer), ethanol, polyol, such as glycerol, propylene glycol, polyethylene glycol, suitable mixtures thereof, surfactants or vegetable oils. A compound of the invention can also be formulated into liposomes, in particular for parenteral administration. Liposomes provide the advantage of increased half-life in the circulation, if compared to the free drug and a prolonged even more release of the enclosed drug.

Production of sterile injectable solutions containing one or several of the compounds of the invention is accomplished by incorporating the respective compound in the required amount in the appropriate solvent with various ingredients enumerated above as required followed by sterilization. To obtain a sterile powder the above solutions are vacuum-dried or freeze-dried as necessary. Preferred diluents of the present invention are water, physiologically acceptable buffers, physiologically acceptable buffer salt solutions or salt solutions. Preferred carriers are cocoa butter and vitebesole.

Further excipients which can be used with the various pharmaceutical forms of a compound of the invention can be chosen from the following non-limiting list:
a) binders such as lactose, mannitol, crystalline sorbitol, dibasic phosphates, calcium phosphates, sugars, microcrystalline cellulose, carboxymethyl cellulose, hydroxyethyl cellulose, polyvinyl pyrrolidone and the like;
b) lubricants such as magnesium stearate, talc, calcium stearate, zinc stearate, stearic acid, hydrogenated vegetable oil, leucine, glycerids and sodium stearyl fumarates,
c) disintegrants such as starches, croscaramellose, sodium methyl cellulose, agar, bentonite, alginic acid, carboxymethyl cellulose, polyvinyl pyrrolidone and the like.

Other suitable excipients can be found in the Handbook of Pharmaceutical Excipients, published by the American Pharmaceutical Association, which is herein incorporated by reference.

Further embodiment of the invention relates to the compound for use of the present invention or to the metabolite of the compound of the present invention for use of the present invention, wherein the treatment dosage is from 1 mg/day to 1000 mg/day. In a further embodiment, the treatment dosage is from 1 mg/day to 1000 mg/day. The lower limits are for instance 1 mg/day, 5 mg/day, 10 mg/day, 20 mg/day, 25 mg/day or 50 mg/day. The upper limits are for instance 1000 mg/day, 900 20 mg/day, 800 mg/day, 750 mg/day, 700 mg/day, 600 mg/day, 500 mg/day, 250 mg/day, 200 mg/day. It is to be understood that each upper limit can be combined with each lower limit. In a preferred embodiment, the dosage is from 10 mg/day to 200 mg/day.

In a further embodiment, the present invention relates to the pharmaceutical composition for use of the present invention, wherein the compound of formula (I) or the metabolite of the compound of the formula (I) is to be dosed in an amount of 1 mg/day to 1000 mg/day. The lower limits are for instance 1 mg/day, 5 mg/day, 10 mg/day, 20 mg/day, 25 mg/day or 50 mg/day. The upper limits are for instance 1000 mg/day, 900 mg/day, 800 mg/day, 750 mg/day, 700 mg/day, 600 mg/day, 500 mg/day, 250 mg/day, 200 mg/day. It is to be understood that each upper limit can be combined with each lower limit. In a preferred embodiment, the dosage is from 10 mg/day to 200 mg/day.

As understood herein, the compound for use of the present invention, the metabolite of the compound of formula (I) for use of the present invention or the pharmaceutical composition for the use of the present invention can be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

It is noted that the compound of formula (I), in particular tripentadecanoin, can be obtained from herbs or from human/animal milk. The compound of the present invention can be obtained from the plants of genus Ophioglossum. Thus, in a further embodiment, the present invention relates to Ophioglossum for use in the treatment, prevention and/or alleviation of symptoms of neurological complication(s) of a viral infection disease.

In the present invention, it is to be understood that Ophioglossum comprises all species of Ophioglossum. In particular, it consists of the group selected from Ophioglossum sp., Ophioglossum L., Ophioglossum thermale Kom., Ophioglossum thermale Komarov, Ophioglossum austro-asiaticum Nishida, Ophioglossum austroasiaticum Nish., Ophioglossum petiolatum L., Ophioglossum pendulum L., Ophioderma pendula (L.) Presl., Ophioglossum reticulatum L., Ophioglossum vulgatum L., Ophioglossum pedunculosum Desv., Ophioglossum parvifolium Grey. et HK., Ophioglossum petiolatum Hook., Ophioglossum petiolatum Hooker, Ophioglossum 25 tenerum, Ophioglossum pycnostichum, Ophioglossum pycnostichum (Fern.) A.&D. Love, Ophioglossum pycnostichum (Fernald) A.Löve & D.Löve; O. vulgatum var. pycnostichum Fernald, Ophioglossum crotalophoroides Walt., Ophioglossum crotalophoroides Walter var. crotalophoroides, Ophioglossum crotalophoroides Walter var. nanum Osten ex J. S. Licht., Ophioglossum azoricum, Ophioglossum azoricum C. Presl, Ophioglossum vulgatum Linnaeus var. pseudopodum (S. F. Blake) Farwell, Ophioglossum dendroneuron E. P. St.John; O. ellipticum Hooker & Greville; O. mononeuron E. P. St.John, Ophioglossum dendroneuron E. P. St. John, Ophioglossum Linnaeus, Ophioglossum palmatum L., 5 Ophioglossum mononeuron E. P. St. John, Ophioglossum austroasiaticum, Ophioglossum bergianum, Ophioglossum bucharicum, Ophioglossum californicum, Ophioglossum caroticaule, Ophioglossum convexum, Ophioglossum californicum Prantl, Ophioglossum concinnum, Ophioglossum concinnum Brack., Ophioglossum costatum, Ophioglossum costatum R.Br., Ophioglossum coriaceum, Ophioglossum decipiens, Ophioglossum dietrichiae, Ophioglossum dudadae, Ophioglossum engelmannii, Ophioglossum engelmannii Prantl, Ophioglossum ellipticum Hook. & Grey., Ophioglossum fernandezianum, Ophioglossum gomezianum, Ophioglossum gracile, Ophioglossum gramineum Willd., Ophioglossum gramineum, Ophioglossum harrisii, Ophioglossum intermedium, Ophioglossum kawamurae, Ophioglossum lancifolium, Ophioglossum latifolium, Ophioglossum litorale, Ophioglossum loureirianum, Ophioglossum lusitanicum L., Ophioglossum lusitanicum L. ssp. californicum (Prantl) R. T. Clausen, Ophioglossum lusitanicum L. var. californicum (Prantl) Broun, Ophioglossum moultoni, Ophioglossum namegatae, Ophioglossum nudicaule, Ophioglossum nudicaule L. f., Ophioglossum nudicaule L. f. var. minus R. T. Clausen, Ophioglossum nudicaule L. f. var. tenerum (Mett. ex Prantl) R. T. Clausen, Ophioglossum oblongum, Ophioglossum obovatum, Ophioglossum opacum, Ophioglossum ovatum, Ophioglossum parvifolium, Ophioglossum parvum, Ophioglossum pendulum, Ophioglossum pendulum L. ssp. falcatum (C. Presl) R. T. Clausen, Ophioglossum pendulum L. ssp. Pendulum, Ophioglossum petiolatum, Ophioglossum polyphyllum, Ophioglossum polyphyllum A. Braun, Ophioglossum polyphyllum A. Braun ex Schub., Ophioglossum pumilio, Ophioglossum pusillum, Ophioglossum pusillum Raf., Ophioglossum raciborskii, Ophioglossum ramosii, Ophioglossum reticulatum, Ophioglossum rubellum, Ophioglossum savatieri, Ophioglossum scariosum, Ophioglossum schm idii, Ophioglossum simplex, Ophioglossum thermal, Ophioglossum thomasii, Ophioglossum timorense, Ophioglossum tenerum Mett. ex Prantl, Ophioglossum usterianum, Ophioglossum vulgatum, Ophioglossum vulgatum. auct. non L., Ophioglossum vulgatum L. var. alaskanum (E. G. Britton) C. Chr., Ophioglossum vulgatum L. var. pseudopodum (S. F. Blake) Farw., Ophioglossum vulgatum L. var. pycnostichum Fernald, Ophioglossaceae Martinov, Cheiroglossa palmata (L.) C. Presl, Ophioglossum eliminatum Khand. & Goswami, Ophioglossum namegatae Nish. & Kurita, Ophioglossum nipponicum Miyabe & Kudô, and/or Ophioglossum oleosum Khand.

Preferred Ophioglossum are Ophioglossum thermale, Ophioglossum petiolatum, Ophioglossum reticulatum, Ophioglossum parvifolium, Ophioglossum vulgatum, Ophioglossum austroasiaticum, Ophioglossum azoricum, Ophioglossum californicum, Ophioglossum costatum, Ophioglossum crotalophoroides, Ophioglossum engelmanii, Ophioglossum lusitanicum, Ophioglossum nudicaule, Ophioglossum polyphyllum, Ophioglossum pusillum, and/or Ophioglossum pycnosticum. Particularly preferred Ophioglossum are Ophioglossum thermale, Ophioglossum petiolatum, Ophioglossum reticulatum, Ophioglossum vulgatum, and/or Ophioglossum austro-asiaticum Nishida.

The most preferred Ophioglossum is Ophioglossum thermale.

One embodiment of the invention relates to the ophioglossum for use of the present invention in an amount (herein understood as a dosing regime) that corresponds to administering 1 mg/day to 1000 mg/day of the compound of formula (I), preferably of tripentadecanoin.

A further embodiment relates to the ophioglossum for use of the present invention in an amount that corresponds to administering the compound compound of formula (I), preferably of tripentadecanoin, from 1 mg/day to 1000 mg/day. The lower limits are for instance 1 mg/day, 5 mg/day, 10 mg/day, 20 mg/day, 25 mg/day or 50 mg/day of the compound of the formula (I), preferably of tripentadecanoin, contained in ophioglossum. The upper limits are for instance 1000 mg/day, 900 mg/day, 800 mg/day, 750 mg/day, 700 mg/day, 600 mg/day, 500 mg/day, 250 mg/day, 200 mg/day of the compound of the formula (I), preferably of tripentadecanoin, contained in ophioglossum. It is to be understood that each upper limit can be combined with each lower limit. In a preferred embodiment, the dosage is from 10 mg/day to 200 mg/day.

Alternatively, the present invention relates to ophioglossum for use of the present invention, wherein the ophioglossum is administered to a subject/patient in an amount of 10 mg to 10000 mg/day of dry ophioglossum powder. The lower limits are for instance 10 mg/day, 20 mg/day, 30 mg/day, 40 mg/day, 50 mg/day, 100 mg/day, 150 mg/day, 200 mg/day, 300 mg/day, 500 mg/day, 700 mg/day. The upper limits are for instance 10000 mg/day, 8000 mg/day, 6000 mg/day, 5000 mg/day, 2500 mg/day, 1000 mg/day. It is to be understood that each upper limit can be combined with each lower limit.

In a further embodiment, the present invention relates to a pharmaceutical composition for the use in the treatment, prevention and/or alleviation of symptoms of neurological complication(s) of a viral infection disease, wherein the composition contains ophioglossum in an amount that corresponds to administering 1 mg/day to 1000 mg/day of the compound of the formula (I), preferably of tripentadecanoin.

Alternatively, the invention relates to the pharmaceutical composition for use, wherein ophioglossum is to be administered in the amount of 10 mg to 10000 mg/day of dry ophioglossum powder. The lower limits are for instance 10 mg/day, 20 mg/day, 30 mg/day, 40 mg/day, 50 mg/day, 100 mg/day, 150 mg/day, 200 mg/day, 300 mg/day, 500 mg/day, 700 mg/day. The upper limits are for instance 10000 mg/day, 8000 mg/day, 6000 mg/day, 5000 mg/day, 2500 mg/day, 1000 mg/day. It is to be understood that each upper limit can be combined with each lower limit.

The ophioglossum for use of the present invention preferably in an amount as indicated herein, as well as the pharmaceutical composition of the present invention comprising ophioglossum as described herein, can be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The compound for use of the present invention, the metabolite of the compound for use of the present invention for use of the present invention, the pharmaceutical composition for use of the present invention, or the ophioglossum for use of the present invention are particularly useful in the treatment, prevention and/or alleviation of symptoms of neurological complication(s) of a viral infection disease.

Within the scope of the present invention, any viral infection disease that may lead to neurological complications, in particular neurological complications that are symptomatic, is encompassed. The virus infection disease can be due to a DNA virus (double or single stranded), an RNA virus (single or double stranded, whether positive of negative), a reverse transcribing virus or any emerging virus, whether enveloped or non-enveloped.

In certain embodiments of the invention, the virus infection disease is related to a respiratory virus infection. Preferably, the respiratory virus as described herein is a virus selected from the group of Rhinovirus, RSV, Parainfluenza, Metapneumovirus, Coronavirus, Enterovirus, Adenovirus, Bocavirus, Polyomavirus, Herpes simplex virus, and Cytomegalovirus.

In certain embodiments of the invention, the virus infection disease is related to a DNA virus infection. Preferably, the DNA virus as described herein is selected from the group consisting of Adenovirus, Rhinovirus, RSV, Influenza virus, Parainfluenza virus, Metapneumovirus, Coronavirus, Enterovirus, Adenovirus, Bocavirus, Polyomavirus, Herpes simplex virus, Cytomegalovirus, Bocavirus, Polyomavirus, and Cytomegalovirus.

In certain embodiments of the invention, the virus infection disease is related to an RNA virus infection. The RNA virus may be an enveloped or coated virus or a nonenveloped or naked RNA virus. The RNA virus may be a single stranded RNA (ssRNA) virus or a double stranded RNA (dsRNA) virus. The single stranded RNA virus may be a positive sense ssRNA virus or a negative sense ssRNA virus. Preferably, the RNA virus as described herein is selected from the group consisting of Rhinovirus, RSV, Influenza virus, Parainfluenza virus, Metapneumovirus, Coronavirus, Enterovirus Adenovirus, Bocavirus, Polyomavirus, Herpes simplex virus, and Cytomegalovirus.

In certain embodiments of the present invention, the virus infection disease is related to a Coronavirus infection. Preferably, the Coronavirus as described herein is a Coronavirus from the genus selected from the group of α-CoV, β-CoV, γ-CoV and δ-CoV. Further preferably, the Coronavirus as described herein is of the genus α-CoV or β-CoV. In certain embodiments, the Coronavirus as described herein is selected from the group consisting of Human coronavirus OC43 (HCoV-OC43), Human coronavirus HKU1 (HCoV-HKU1), Human coronavirus 229E (HCoV-229E), Human coronavirus NL63 (HCoV-NL63, New Haven coronavirus), Middle East respiratory syndrome-related coronavirus (MERS-CoV or “novel coronavirus 2012”), Severe acute respiratory syndrome coronavirus (SARS-CoV or “SARS-classic”), and Severe acute respiratory syndrome coronavirus 2 (SARS-CoV-2 or “novel coronavirus 2019”).

Preferably, the virus infection is an RNA virus infection, most preferably a coronavirus infection due to a coronavirus selected from the non-limiting group comprising MERS-CoV, SARS-CoV and SARS-CoV-2. Most preferably the virus infection is a SARS-CoV-2 infection.

In one embodiment within the scope of the present invention, the virus infection disease is caused by SARS-CoV-2, SARS-CoV-1, MERS, influenza virus, human immunodeficiency virus (HIV), varicella-zoster virus (VZV), herpes simplex virus (HSV), poliovirus, Epstein-Barr virus (EBV) cytomegalovirus (CMV), Japanese Encephalitis virus, Venezuelan Equine Encephalitis virus, California encephalitis virus or zika virus. Preferably, the virus infection is an RNA virus infection, most preferably a coronavirus infection due to a coronavirus selected from the non-limiting group comprising MERS-CoV, SARS-CoV and SARS-CoV-2. Most preferably the virus infection is a SARS-CoV-2 infection. Thus, in a preferred embodiment of the present invention, the virus infection disease is caused by a coronavirus, preferably by SARS-CoV-2, SARS-CoV-1, or MERS. More preferably, the virus infection disease is caused by SARS-CoV-2. SARS-CoV-2 is known to cause a virus infection disease referred to as COVID-19.

The virus infection disease caused by a variant of the virus as described herein is also encompassed by the present invention. In particular, the variant of SARS-CoV-2 is selected from the group consisting of Lineage B.1.1.207, Lineage B.1.1.7, Cluster 5, 501.V2 variant, Lineage P.1, Lineage B.1.429/CAL.20C, Lineage B.1.427, Lineage B.1.526, Lineage B.1.525, Lineage B.1.1.317, Lineage B.1.1.318, Lineage B.1.351, Lineage B.1.617 and Lineage P.3. In certain embodiments, the SARS-CoV-2 variant described herein is a SARS-CoV-2 variant described by a Nextstrain Glade selected from the group consisting of 19A, 20A, 20C, 20G, 20H, 20B, 20D, 20F, 201, and 20E. In certain embodiments, the SARS-CoV-2 virus described herein is a SARS-CoV-2 variant comprising at least one mutation of the spike protein selected from the group consisting of D614G, E484K, N501Y, S477G/N, P681H, E484Q, L452R and P614R. In certain embodiments of the present invention, the SARS-CoV-2 variant described herein is a SARS-CoV-2 variant derived from the variants described herein. In certain embodiments, the SARS-CoV-2 virus described herein is a SARS-CoV-2 variant having an at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% sequence identity to the viral genome sequence of at last one SARS-CoV-2 variant described herein.

Any conceivable neurological complication of a viral infection disease can be treated by using the compound for use of the present invention, the metabolite of the compound for use of the present invention for use of the present invention, the pharmaceutical composition for use of the present invention, or the ophioglossum for use of the present invention.

In one embodiment, the neurological complication of a viral infection disease is damage of the central nervous system. In particular, the neurological complication of a viral infection disease is damage of the brainstem. The damage of the brainstem may manifest itself in several different ways. In particular, the damage of the brainstem has been shown to occur as a complication of the viral infection disease caused by SARS-CoV-2.

In certain embodiments, treating, preventing and/or alleviating the symptoms of a viral infection disease constitutes treating, preventing and/or alleviating the symptoms of a brainstem damage.

The part of the brainstem that is commonly attacked by the virus is ventolateral medulla, part of medulla oblongata of the brainstem which plays a major role in regulating arterial blood pressure and/or breathing. In other words, within the scope of the present invention the damage to the central nervous system may comprise damage to ventolateral medulla. In certain cases, the virus can invade the central nervous system, attack the brainstem's nerve center that controls breathing and heartbeat, as discussed herein, and cause asymptomatic sudden death. Therefore, in certain embodiments preventing the neurological complications of the viral infection disease constitutes preventing asymptomatic sudden death.

The virus can also cause a cardiorespiratory failure. Further symptoms of the damage of the central nervous system, in particular of brainstem, are difficulties in breathing and/or in heartbeat. Thus, in certain embodiments, treating, preventing and/or alleviating the symptoms of a viral infection disease constitutes treating, preventing and/or alleviating the symptoms of difficulties in breathing and/or in heartbeat.

In certain embodiments, the damage of the central nervous system may comprise the damage to the solitary nucleus. The solitary nucleus is a series of purely sensory nuclei (clusters of nerve cell bodies) forming a vertical column of grey matter embedded in the medulla oblongata. Inputs of the solitary nucleus include taste information from the facial nerve via the chorda lympani, glossopharyngeal nerve and vagus nerve, chemoreceptors and mechanoreceptors of the general viscera afferent pathway (GVA) in the carotid body via glossopharyngeal nerve, aortic bodies, and the sinoatrial node, via the vagus nerve, as well as chemically and mechanically sensitive neurons of the GVA with endings located in the heart, lungs, airways, gastrointestinal system, pharynx, and liver via the glossopharyngeal and vagus nerves. Further input includes input from the nasal cavity, soft palate and sinus cavities via the facial nerve. As known to the skilled person neurons that innervate the solitary nucleus mediate the gag reflex, the carotid sinus reflex, the aortic reflex, the cough reflex, the baroreceptor reflex and chemoreceptor reflex, as well as respiratory reflexes and reflexes within the gastrointestinal systems that regulate the motility and secretion. The outputs include paraventricular nucleus of the hypothalamus and the central nucleus of the amygdala, as well as other nuclei in the brainstem. Any of these processes may be interfered with upon infection with the virus and onset of the viral infectious disease. In particular, the symptoms may include anosmia (also referred to as smell blindness), dysphagia (which is understood to comprise swallowing difficulties), and/or further garstroinstestinal difficulties.

In certain embodiments of the present invention, treating, preventing and/or alleviating the symptoms of the neurological complication(s) of a viral infection disease constitutes treating, preventing and/or alleviating the symptoms of the damage to the solitary nucleus. Further treating, preventing and/or alleviating the symptoms of the neurological complication(s) of a viral infection disease constitutes treating, preventing and/or alleviating anosmia (also referred to as smell blindness), dysphagia (which is understood to comprise swallowing difficulties), and/or further gastrointestinal difficulties.

In certain embodiments, the damage of the central nervous system may comprise the damage to the dorsal vagal nucleus, located within the medulla oblongata of the brain stem. Dorsal vagal nucleus serves functions in the gastrointestinal tract, lungs as well as thoracic and abdominal innervations. Damage to the dorsal vagal nucleus may result in, among others, cardiorespiratory failure. Thus, in certain embodiments of the present invention, preventing the neurological complication(s) of a viral infection disease constitutes preventing cardiorespiratory failure.

In certain embodiments, the damage of the central nervous system may comprise the damage to the olfactory bulb. As understood herein, the damage to the olfactory bulb may result in loss of smell and/or loss of taste. Thus, in certain embodiments of the present invention, treating, preventing and/or alleviating the symptoms of the neurological complication(s) of a viral infection disease constitutes treating, preventing and/or alleviating the symptoms of the loss of smell and/or the loss of taste.

In certain embodiments of the present invention, the damage of the central nervous system may lead to a neurodegenerative disease. Neurodegenerative disease is the umbrella disease term for the progressive loss of structure or function of neurons, including death of neurons. The damage or death of neurons lead to a gradual deterioration of the functions controlled by the affected part of the nervous system. The selected group of neurodegenerative disorders include Alzheimer's Disease (AD), Parkinson's disease (PD), Huntington's diseases (HD), Amyotrophic Lateral Sclerosis (ALS), Dementia, dementia with Lewy bodies (DB), frontotemporal dementia (FTD), and brain atrophy. Apoptosis, or programmed cell death, plays an important role in both physiologic and pathologic conditions. There is mounting evidence for an increased rate of apoptotic cell death in a variety of acute and chronic neurological diseases including neurodegenerative disease. Apoptosis is characterized by neuronal shrinkage, chromatin condensation, and DNA fragmentation, whereas necrotic cell death is associated with cytoplasmic and mitochondrial swelling followed by dissolution of the cell membrane. Evidence of DNA fragmentation has been found in several degenerative neurologic disorders, including AD, HD and ALS.

In certain embodiments, the damage of the central nervous system may comprise the damage to the substantia nigra. Damage to substantia nigra is known to the skilled person to be associated causatively with the onset of Parkinsons disease.

Parkinson's disease is a degenerative disorder of the central nervous system. It results from the death of dopamine-generating cells in the substantia nigra, a region of the midbrain; the cause of cell-death is unknown. Parkinson's disease is the second most common neurodegenerative disorder and manifests as bradykinesia, rigidity, resting tremor and posture instability. PD affects approximately 7 million people globally and 1 million people in the United States. The number of new cases per year of PD is between 8 and 18 per 100,000 persons—year. Levodopa has been the most widely used treatment for over 30 years but with very limited efficacy. Investigations on neuroprotection are at the forefront of PD research.

Thus, the present invention further relates to an embodiment, wherein treating, preventing and/or alleviating the symptoms of neurological complication(s) of a viral infection disease is treating, preventing and/or alleviating the symptoms of neurodegenerative diseases such as Alzheimer's disease or Parkinson's disease.

In certain embodiments, the damage of the central nervous system may comprise damage to the motor cortex. The damage to the motor cortex may lead to headache, seizures, muscle weakness, malaise and/or fatigue. Thus, the neurological complication(s) of a viral infection disease may comprise headache, seizures, muscle weakness, malaise and/or fatigue. Furthermore, treating, preventing and/or alleviating the symptoms of a viral infection disease may comprise treating, preventing and/or alleviating headache, seizures, muscle weakness, malaise and/or fatigue.

Further in certain embodiments, the neurological complication(s) of a viral infection disease may comprise perivascular encephalitis, interstitial encephalitis, neuronal cell loss, and/or axon degeneration. Thus, treating, preventing and/or alleviating the symptoms of the neurological complication(s) of a viral infection disease may constitute treating, preventing and/or alleviating the symptoms of perivascular encephalitis, interstitial encephalitis, neuronal cell loss, and/or axon degeneration.

Further in certain embodiments, the neurological complication(s) of a viral infection disease may comprise unconsciousness, confusion and/or altered mental status. Thus, treating, preventing and/or alleviating the symptoms of the neurological complication(s) of a viral infection disease may constitute treating, preventing and/or alleviating the symptoms of unconsciousness, confusion and/or altered mental status.

In certain embodiments, the neurological complication(s) of a viral infection disease comprise damage of the peripheral nervous system. Thus, neurological complication(s) of a viral infection disease is preventing the damage of the peripheral nervous system. In certain embodiments, said complication(s) include Guillain Barré syndrome. As known to the skilled person, Guillain Barré syndrome is considered to have been caused by a response of the immune system of a subject to an infection disease, in particular viral infection disease. Guillain Barré syndrome has been associated with infections with cytomegalovirus, Epstein-Barr virus, varicella zoster virus, dengue virus, Zika virus, and Hepatitis virus. Guillain Barré syndrome has also been reported to be associated with SARS-CoV-2 infection. Therefore, in certain embodiments, the neurological complications(s) of a viral infection disease are due to autoimmune response to the viral infection disease. In certain embodiments, said complication(s) include Guillain Barré syndrome.

In certain embodiments, the neurological complication(s) of a viral infection disease comprise an inflammatory disease of the nervous system. As understood herein, the inflammatory disease of the nervous system described herein can be an inflammatory disease of the parasympathetic nervous system, an inflammatory disease of the central nervous system or an inflammatory disease of the peripheral nervous system. In certain embodiments, the inflammatory disease of the nervous system is selected from the group of multiple sclerosis, amyotrophic lateral sclerosis, Alzheimer's disease, Parkinson's disease and Huntington's disease.

As known to the skilled person, viruses hijack the host's cellular machinery to replicate efficiently, in particular by applying diverse strategies to manipulate proteostasis pathways at different stages to take advantage of their cycle progression. Protein homeostasis, also referred to as proteostasis, is the result of coordinated networks that act to maintain a dynamic equilibrium among protein translation, folding, and clearance. It includes molecular chaperones, predominantly the heat shock proteins (which may also be referred to as HSPs), which enable the correct protein folding, native conformation maintenance, and cooperation with the protein degradation machinery. Without wishing to be bound by the theory, it may be postulated that neurological complication(s) of a viral infection disease may be, at least in part, related to interplay between proteostasis and the viral infection disease. It is noted that cells use their potential for degrading and recycling their own components as the means of killing intracellular pathogens, including infecting viruses. For example, autophagy represents an innate immune defense mechanism against viruses by delivering viruses and/or viral proteins to lysosomes for degradation. In turn, viruses have developed mechanisms to interfere with protein degradation pathways to maintain the correct concentration and function of viral proteins.

Further without wishing to be bound by the theory, it is noted that the compound of formula (I), preferably tripentadecanoin, is useful in treating, preventing and/or alleviating the symptoms of the neurological complication(s) of a viral infection disease through affecting the expression of neuroglobin protein.

Expression of neuroglobin has demonstrated to be neuroprotective, neuro rescuing and anti-apoptotic. As it is known to the skilled person, neuroglobin is downregulated in several neurodegenerative diseases/models and its downregulation correlates with poor prognosis. In turn, over-expression of neuroglobin showed therapeutic effects in disease models of neurodegenerative disorders. Neuroglobin is an intracellular heme protein expressed in the central and peripheral nervous system, cerebrospinal fluild, retina and endocrine tissues. The heme group of the protein coordinates an Fe(III) ion. During cerebral ischemia, neuroglobin becomes able to bind O2 with higher affinity than in normal conditions due to the fact that the heme-Fe atoms shifts from the ferric (Fe(III)) to the ferrous (Fe(II)) form. Neuroglobin has been shown to scavenge the reactive oxygen and nitrogen species in neurons. More importantly, neuroglobin has been shown to inhibit the intrinsic apoptosis pathways, binding to Mitochondrial permeability transition pores (MPTP) component (VDAC) and preventing cytochrome c release. Thus, neuroglobin is an essential protein for regulating neuronal survival. Neuroglobin was mainly (˜90%) localized in the cytosol, but accumulating evidence revealed that it is also associated with mitochondria. Neuroglobin is a particularly highly conserved protein, with mouse and human neuroglobins differing in only 6% of the amino acid positions.

In vivo experiments have shown that increased levels of neuroglobin significantly protect both heart and brain from hypoxic/ischemic and/or oxidative stress-related insults, whereas decreased neuroglobin levels may lead to an exacerbation of tissue injuries. Thus, in certain embodiments the neurological complication(s) of a viral infection disease comprise hypoxia, hypoxic/ischemic and/or oxidative stress-related insult. Human neuroglobin overexpression has been hypothesized to protect neurons from mitochondrial dysfunctions and neurodegenerative disorders such as Alzheimer's disease, and to play a shielding role in cancer cells.

Neuroglobin, is herein understood preferably as a protein of sequence identity of at least 90% to a sequence as in SEQ ID NO: 1, more preferably neuroglobin is a protein of sequence identity of at least 90% to a sequence as in SEQ ID NO: 1, even more preferably neuroglobin is a protein of sequence as in SEQ ID NO: 1. SEQ ID. NO: 1 is a sequence of human (Homo sapiens) neuroglobin. For reference purposes, a sequence of mouse (Mus musculus) neuroglobin is as in SEQ ID NO.: 2.

The present inventors have surprisingly found that upon treatment of the yeast cells with the compound of formula (I), herein tripentadecanoin, expression of YHB1, a yeast homologue of neuroglobin is significantly increased (see Example 3 for details). The present inventors have further surprisingly found that upon treatment of cells with the compound of formula (I), herein tripentadecanoin, the aggregation of HSP-104 (observed through GFP-HSP104 loci) is greatly reduced. However, this effect is not observable in the cells wherein YHB1, a homologue of neuroglobin, is knocked out (see Example 3 for details). Without wishing to be bound by the theory, it has been demonstrated that the treatment with the compound of formula (I), in particular with tripentadecanoin, delays the formation of p-bodies in the cells, and postulated that the compound of formula (I), in particular tripentadecanoin, targets p-bodies regulation of YHB1 mRNA decay in budding yeast, and may target p-body regulation of neuroglobin mRNA decay in human.

Further unexpectedly, the present inventors have shown that increased expression of neuroglobin is observed upon treatment of the mouse neurons with the compound of formula (I), herein tripentadecanoin. As presented in Example 4, approximately six-fold increase in the expression level of neuroglobin has been observed.

Therefore, the present invention relates to an embodiment, wherein the compound of formula (I) upon administration to the subject upregulates expression of neuroglobin, preferably in neuron cells. Preferably, the present invention relates to an embodiment, wherein upregulated expression of neuroglobin inhibits apoptosis of neuron cells.

As known to the skilled person, a subject (a patient) suffering from neurological complication(s) of a viral infection disease (in particular the viral infection disease caused by SARS-CoV-2) could benefit from increased expression of neuroglobin in the neuron cells. However, to date it is not obvious to a skilled person how the expression of neuroglobin could be increased in the neuron cells of said subject (patient). It is noted that unexpected discovery of the link between the administration of the compound according to formula (I) and the expression of neuroglobin in the neuronal cells allowed qualifying subject(s) (patient(s)) suffering from neurological complication(s) of a viral infection disease for treatment with the compound of formula (I) as described herein, its metabolite, the pharmaceutical composition as described herein of ophioglossum as described herein.

In a further embodiment, the present invention relates to the compound of formula (I) as defined herein, or to ophioglossum as defined herein, for use as a functional food for treatment, prevention and/or alleviation of symptoms of neurological complication(s) of a viral infection disease, as defined hereinabove.

Functional food refers to a food given an additional function (often one related to health-promotion or disease prevention) by adding new ingredients or enriching existing ingredients. The term may also apply to traits purposely bred into existing edible plants, such as purple or gold potatoes having enriched anthocyanin or carotenoid contents, respectively. Functional foods may be “designed to have physiological benefits and/or reduce the risk of chronic disease beyond basic nutritional functions, and may be similar in appearance to conventional food and consumed as part of a regular diet” (US Department of Agriculture, Agricultural Research Service, AgResearch Magazine. November 2014; US Department of Agriculture, Agricultural Research Service. July 2010).

In a further embodiment, the present invention relates to the compound of formula (I) as defined herein, or to ophioglossum as defined herein, for use as a dietary supplement for treatment, prevention and/or alleviation of symptoms of neurological complication(s) of a viral infection disease, as defined herein.

In a further embodiment, the present invention relates to a non-therapeutic use of a functional food or dietary supplement comprising the compound of formula (I) as defined herein, or comprising ophioglossum, as defined herein, to upregulate expression of neuroglobin in a subject, preferably in neuron cells. As understood herein, upregulated expression of neuroglobin inhibits apoptosis of neuron cells.

In a further embodiment of the present invention, functional food for human and/or animals, comprises the compound of formula (I), preferably tripentadecanoin at the dosage from 1 μg (microgram)/day to 50 mg/day, preferably at the dosage from 1 μg (microgram)/day to 20 mg/day. The lower limits are for instance 1 μg (microgram)/day, 2 μg (microgram)/day, 3 μg (microgram)/day, 4 μg (microgram)/day, 5 μg (microgram)/day, 7 μg (microgram)/day, 10 μg (microgram)/day, 20 μg (microgram)/day, 25 μg (microgram)/day, 50 μg (microgram)/day, 100 μg (microgram)/day, 200 μg (microgram)/day, 300 μg (microgram)/day, 400 μg (microgram)/day or 500 μg (microgram)/day. The upper limits are for instance 50 mg/day, 40 mg/day, 30 mg/day, 20 mg/day, 10 mg/day, 5 mg/day, 3 mg/day, 2 mg/day, 1 mg/day, 900 μg (microgram)/day. It is to be understood that each upper limit can be combined with each lower limit. In one embodiment, the dosage is from 1 μg (microgram)/day to 20 mg/day. In another embodiment, the dosage is from 1 μg (microgram)/day to 900 μg (microgram)/day.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The invention is illustrated by the following examples, which however are not meant to be construed as limiting.

### EXAMPLES

### Example 1

Tripentadecanoin was tested in a mouse model of coronavirus infection. The objective of this study was to investigate tripentadecanoin's therapeutic effects in MHV infected mice. Mouse hepatitis virus (MHV) causes acute infections of the murine liver and lungs and persistent infections of the gastrointestinal tract and central nervous system (CNS). This virus initially caused hindlimb paralysis, but on repeated passage through mice, more virulent variants, which predominantly caused encephalitis, were selected. This virus was named JHM virus (JHMV) and subsequently shown to be a coronavirus, related to other MHV strains (Bergmann, C., Lane, T. & Stohlman, S. Coronavirus infection of the central nervous system: host-virus stand-off. Nat Rev Microbiol 2006; 4:121-132.). JMHV is now broadly used to study virus-induced neurological diseases, particularly demyelination. It is considered to be a suitable model for coronavirus-induced neurological complication. CNS infection with MHV provides an animal model for acute viral-induced encephalomyelitis and the human demyelinating disease, multiple sclerosis. MHV-induced demyelinating disease is typified by mice, which control viral replication, but fail to fully clear virus. Acute infection induces CNS inflammation and initiates the demyelinating process. Animals that fail to control virus replication in a sufficiently rapid manner succumb to encephalomyelitis within 5-10 days. Survivors of acute infection typically eliminate infectious virus within two weeks. However, these animals still fail to achieve sterile immunity as evidenced by the persisting viral antigen (Ag) and RNA within the CNS. MHV persistence is associated with chronic CNS inflammation and ongoing primary demyelination.

20 C57BL/6J male mice of at 5 weeks of age were randomized into 2 groups of vehicle control or tripentadecanoin 50 mg/kg. Tripentadecanoin or vehicle control treatment was started 3 days after virus infection for a total of 19 days till Day 21. Clinical score of MHV infected mice was rated as: 0—asymptomatic; 1—limp tail; 2—wobbly gait with righting difficulty; 3—hind limb weakness and extreme righting difficulty; 4—hind limb paralysis; 5—moribund.

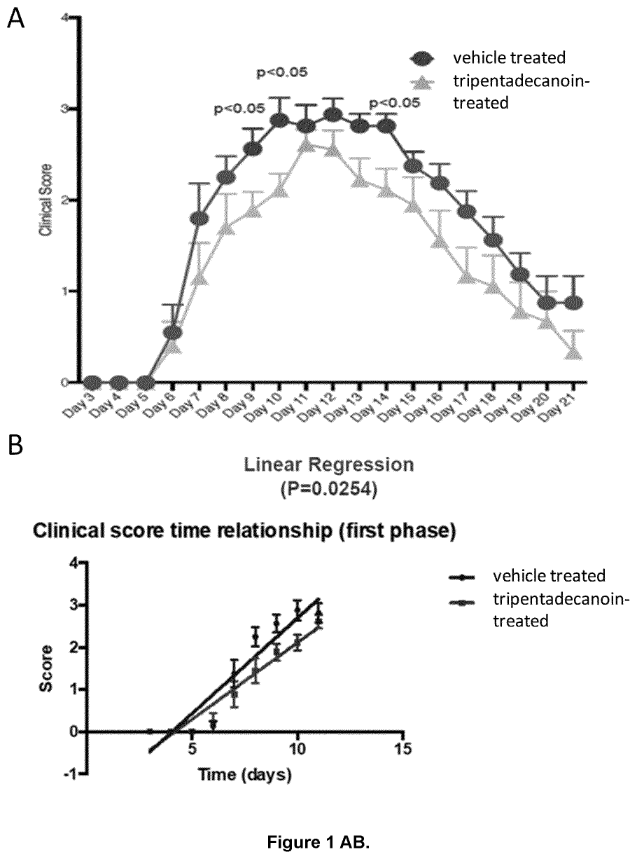

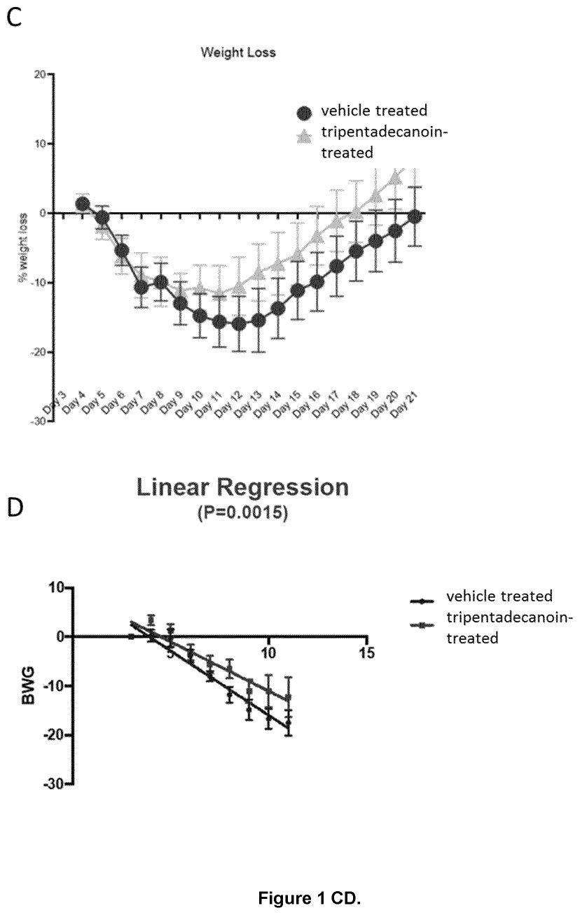

The experimental results are presented in FIG. 1. In this pilot MHV infected mouse study with small sample size, tripentadecanoin showed an encouraging neurorescuing effect in reducing the clinical severity of virus induced neuropathology (P<0.05 on days 9, 10 or 14) (FIG. 1). A linear regression analysis during the first phase showed a statistical significance between the two groups (FIG. 1).

### Example 2

Tripentadecanoin, a representative compound of the compound of formula (I) shows neuroprotective effects in the murine model of N-nitroso-N-methylurea (NMU) photoreceptor degeneration.

Eighteen 8-12-week-old female C57BL/6J mice were randomized into three groups: 1) NMU+vehicle; 2) NMU+Tripentadecanoin 20 mg/kg; 3) NMU+Tripentadecanoin 50 mg/kg. NMU at a dose of 50 mg/kg was injected i.p. to all mice; Tripentadecanoin (or vehicle) was administered daily via oral gavage starting 3 days before NMU and continuing until 7 days after NMU challenge. Retina were imaged by spectral domain optical coherence tomography (OCT) 5 days before exposing to NMU and 7 days after NMU. All eyes were collected and processed for histological hematoxylin and eosin (H&E) staining. The thicknesses of photoreceptors were measured from different locations on OCT images and H&E slides, respectively. Photoreceptor thickness on H&E slides were quantified using automated Leica software (Leica, Heerbrugg), operated by a single masked observer. Results were expressed as the outer nuclear layer (ONL) area under the curve (AUC −1.75 to +1.75 μm) and number of rows of photoreceptor nuclei.

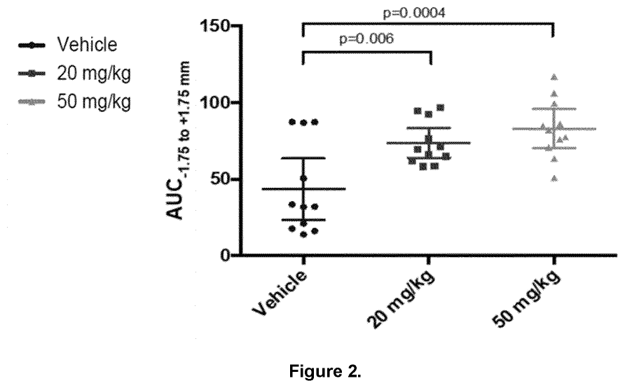

Results: Histological analysis showed that the AUC under the ONL was significantly increased in tripentadecanoin treated groups vs vehicle control, i.e. 83.08±19.05 in 50 mg/kg (p<0.001), 73.58±14.45 in 20 mg/kg (p<0.01) and 43.46±29.86 in vehicle control (FIG. 2). The number of rows of photoreceptor nuclei was significantly increased in tripentadecanoin treated groups vs vehicle control across all regions from central to peripheral, e.g. in mid-peripheral region 7.0±1.7 in 50 mg/kg (p<0.001), 6.4±1.2 in 20 mg/kg (p<0.01) vs 3.8±2.5 in vehicle control. In vivo OCT images showed increased thickness of photoreceptor layers vs control; and neuroretinal thickness in 50 mg/kg tripentadecanoin group was significantly increased in peripheral (p<0.05), mid-peripheral (p<0.05) and central retina (p<0.01) vs vehicle control.

Conclusion: tripentadecanoin exhibits a potent dose-dependent neuroprotective effect against NMU-induced neural (herein: retina) degeneration.

### Example 3

While yeast populations can be propagated ad libitum, the yeast mother cell has a finite lifespan and can only produce a certain amount of daughter cells, typically 25 for laboratory backgrounds. Mortality rate increases with the number of daughter cells produced, therefore this phenomenon is termed replicative ageing. In old yeast cells, age-induced protein deposits recruit a specific set of chaperones and co-chaperones.

Hsp104 expressed endogenously as a fusion to the green fluorescent protein tag (Hsp104-GFP) forms a focus in old cells. The presence (percentage of cells with an aggregate at a certain age), number (number of foci per cell) and size (fluorescent brightness) can be assessed on hundreds of cells and thus, this gives a good model for assessing the biology of age induced aggregates.

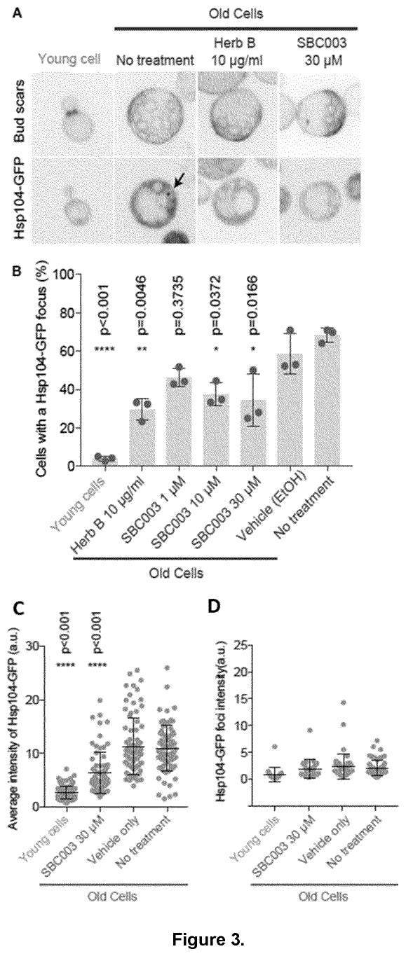

To test if tripentadecanoin is active in yeast, cells were treated with different concentrations of the purified compound (1 μM, 10 μM and 30 μM) as well as an extract (10 μg/ml) of the plant it is derived from (Ophioglossum thermale). These cells were let to age in the presence of the compound and after 10-11 generations, cells were imaged to count their age and assess whether they contained a Hsp104-GFP age-induced protein deposit or not (FIG. 3A). Both the herb extract and tripentadecanoin treated cells that are the same age as untreated cells (vehicle only, 0.3% ethanol) were discovered to were less likely to harbor a Hsp104-GFP focus. To obtain statistical validation of these results, more than 75 cells in each condition were analyzed (FIG. 3B). This result was further confirmed by focusing on one concentration of tripentadecanoin (30 μM) and analyzing more than 500 cells in three independent experiments (FIG. 3CD). Therefore, tripentadecanoin was shown to be a potent effector against one of the major ageing factors identified in yeast cells and conserved in most if not all eukaryotes.

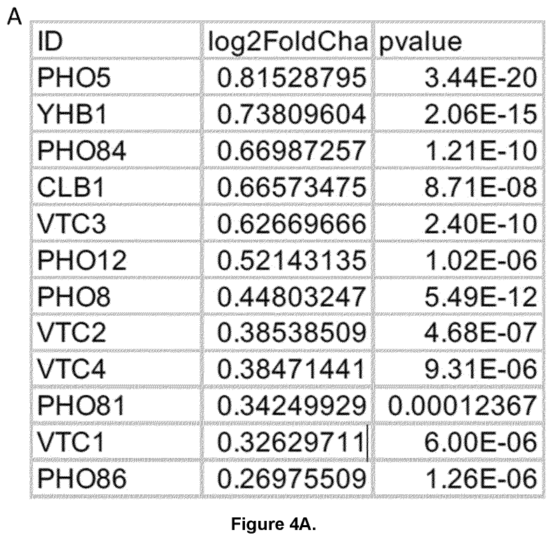

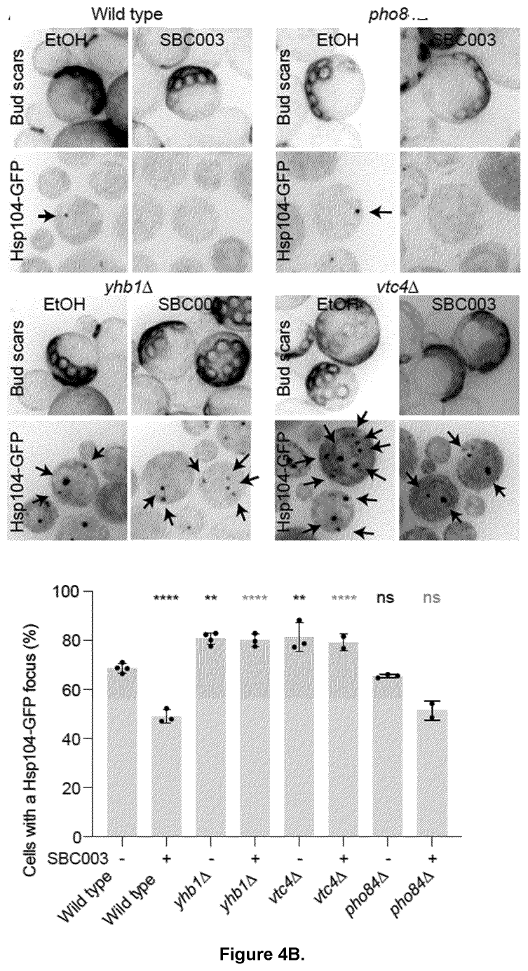

Potential targets of the compound of formula (I) were identified by using RNAseq. The yeast cells (Saccharomyces cerevisiae) were treated with tripentadecanoin (30 μM) for 5 hours and the mRNA abundance of all genes in cells that were treated with the compound or vehicle only for 5 hours was quantitatively monitored. Samples in triplicate were processed by the company Genewiz. 157 mRNAs that were significantly differentially expressed between the two groups were identified. Among these hits is Yhb1, a flavohemoglobin that plays a role in nitrosative stress response (FIG. 4A). Following the results from the RNAseq survey, PH084, YHB1 or VTC4 were knocked out and the proportion of old cells with an Hsp104-GFP focus was measured, as before. While pho84Δ cells were similar to wild type cells, much more vtc4Δ and yhb1Δ mutant cells contained an age-induced deposit (FIG. 4A). The proportion of cells with an Hsp104-GFP focus decreased when wild type and pho84Δ were exposed to tripentadecanoin (30 μM), however, this was not the case for vtc4Δ and yhb1Δ mutant cells. Interestingly, it was noticed that in many vtc4Δ or yhb1Δ mutant old cells, there were not only one but several Hsp104-GFP foci (FIG. 4B). These studies showed that tripentadecanoin relies on the yeast neuroglobin gene to exert its function in yeast natural aging model.

The experimental results are presented in FIG. 4.

### Example 4—Expression of Neuroglobin in Mouse Neurons

Healthy neurons preparation: primary mouse cortical neurons from embryonic day 16-17 were prepared from C57BL/6J mouse foetuses, resulting in 95-97% of neurons and 3-5% astrocytes. At DIV 5/6, mouse primary cortical neurons was treated for 3 h (T3) or 24 h (T24) with vehicle (ethanol) or increasing concentrations of tripentadecanoin (100 or 1000 nM). q PCRmethods used to amplify the mRNA of neuroglobin.

The amount and quality of RNA were evaluated using capillary electrophoresis. The complementary DNA (cDNA) was synthesized by reverse transcription of total RNA in presence of oligo(dT) and “Transcriptor reverse transcriptase” from Roche. The cDNA quantities were then adjusted before the PCRstep. The qPCRreactions were performed using the Licht Cycler system from Roche according to supplier instructions. In this experiment, Rps28 (Ribosomal protein S28) was used as a reference marker (also referred to as housekeeping gene).

The reaction mix (10 μL final) was prepared as follows:
2.5 μL cDNA
Primers and TaqMan probe
Reagent mix containing taq DNA polymerase and MgCl₂.

The following primers were used in this experiment:

Forward primer:
(SEQ ID NO.: 3)
CCCTATCTATGTGTGTCTG
Reverse primer:
(SEQ ID NO.: 4)
TGAGGACCAAGGTATAGA
Probe:
(SEQ ID NO.: 5)
ATCTGCCTGTTGTAGTCTTAGCCTC

The gene coding for neuroglobin was weakly expressed. At 3 hours, tripentadecanoinat 1000 nM treatment induced a significant increased expression of neuroglobin compared to the level of vehicle control (p<0.001). The triplicates are reproducible, and the yield of PCRreaction is similar in the 3 wells of each experimental condition. In addition, the higher concentration of compound according to formula (I) has shown a stronger effect.

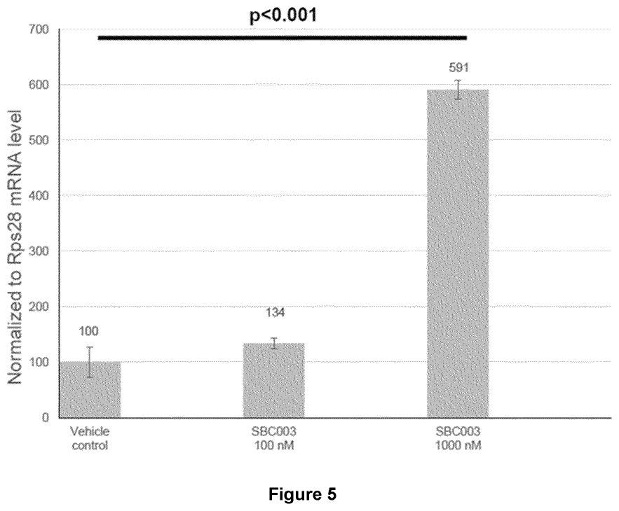

The results of the experiment are shown in FIG. 5.

1. A method for treating, preventing, or alleviating symptoms of a neurological complication of a viral infection disease comprising administering to a subject in need thereof a pharmaceutically effective amount of a compound of formula (I) 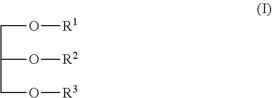
wherein R¹, R² and R³ are independently selected from H or —C(O)—C₁₄-alkyl, provided that at least one of R¹, R² and R³ is —C(O)—C₁₄-alkyl.

2. The method of claim 1, wherein R¹, R² or R³ is —C(O)—C₁₄-alkyl.

3. The method of claim 1, wherein any two of R¹, R² and R³ are —C(O)—C₁₄-alkyl.

4. The method of claim 1, wherein R¹, R² and R³ are —C(O)—C₁₄-alkyl.

5. The method of claim 1, wherein in the compound is tripentadecanoin.

6. A method for treating, preventing, or alleviating symptoms of a neurological complication of a viral infection disease comprising administering to a subject in need thereof a pharmaceutical effective amount of a metabolite of a compound of formula (I) 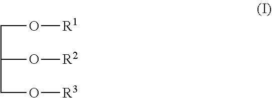
wherein R¹, R² and R³ are independently selected from H or —C(O)—C₁₄-alkyl, provided that at least one of R¹, R² and R³ is —C(O)—C₁₄-alkyl, and the metabolite is HO—C(O)—C₁₄-alkyl or a pharmaceutically acceptable salt thereof.

7. The method of claim 1, wherein the compound of claim 1 is formulated in a pharmaceutical composition containing a pharmaceutically acceptable carrier.

8. A method for treating, preventing, or alleviating symptoms of a neurological complication of a viral infection disease comprising administering to a subject in need thereof a pharmaceutically effective amount of Ophioglossum.

9. The method of claim 1, wherein the viral infection disease is caused by SARS-CoV-2, SARS-CoV-1, MERS, influenza virus, human immunodeficiency virus (HIV), varicella-zoster virus (VZV), herpes simplex virus (HSV), poliovirus, Epstein-Barr virus (EBV) cytomegalovirus (CMV), Japanese Encephalitis virus, Venezuelan Equine Encephalitis virus, California encephalitis virus or zika virus.

10. The method of claim 1, wherein the viral infection disease is caused by SARS-CoV-2, SARS-CoV-1, or MERS.

11. The method of claim 1, wherein the viral infection disease is caused by SARS-CoV-2.

12. The method of claim 1, wherein the neurological complication of a viral infection disease is damage of the central nervous system, in particular of brainstem.

13. The method of claim 1, wherein symptoms of the damage of the central nervous system, in particular of brainstem, are difficulties in breathing or in heartbeat.

14. The method of claim 1, wherein the neurological complication of a viral infection disease comprise damage of the peripheral nervous system.

15. The method of claim 1, wherein symptoms of the damage of the peripheral nervous system are muscle weakness, loss of taste or smell, limb pain, or malaise.

16. The method of claim 1, wherein symptoms of the neurological complication of a viral infection disease are headache or seizures.

17. The method of claim 1, wherein preventing the neurological complication of a viral infection disease is preventing an onset of neurodegenerative diseases such as Alzheimer's disease or Parkinson's disease.

18. The method of claim 1, wherein preventing the neurological complication of a viral infection disease constitutes preventing cardiorespiratory failure.

19. The method of claim 1, wherein the neurological complication of a viral infection disease comprises perivascular encephalitis, interstitial encephalitis, neuronal cell loss, or axon degeneration.

20. The method of claim 1, wherein the compound of formula (I) upon administration to the subject upregulates expression of neuroglobin, preferably in neuron cells.

21. The method of claim 20, wherein upregulated expression of neuroglobin inhibits apoptosis of neuron cells.

22. The method of claim 1, wherein the compound of formula (I) is administered in a dosage from 1 mg/day to 1000 mg/day.
